(19) United States
(12) Patent Application Publication
(10) Pub. No.: US 2007/0282447 A1
(43) Pub. Date: Dec. 6, 2007

(54) Fluid-transfer device, vertebral treatment device,system and methods of use

(76) Inventors: Joseph W. Yedlicka, Indianapolis, IN (US); Robert A. Till, Avon, IN (US); Nancy S. Yedlicka, Indianapolis, IN (US); Patricia M. Till, Avon, IN (US)

Correspondence Address: Mark. S. Leonardo, Esq.;Brown Rudnick Berlack Israels LLP, BOX IP, One Financial Center, Boston, MA, 02111, US

(21) Appl. No.: 11/788,403

(22) Filed: Apr. 20, 2007

Related U.S. Application Data
(60) Provisional application No. 60/809,945, filed on Jun. 1, 2006.

(51) Int. Cl. A61F 2/44; A61B 17/58

(52) U.S. Cl. 623/17.12; 606/86

(57) ABSTRACT

A fluid transfer device is provided, which is adapted to treat a vertebral body. The fluid transfer device includes a first cavity having a first plunger disposed therewith. The first plunger is configured to draw a first fluid into the first cavity. A second cavity has a second plunger disposed therewith. The second plunger is configured to expel a second fluid from the second cavity. An actuator is connected to the first plunger and the second plunger. A bone drill and forceps are provided, which are adapted for treating a vertebral body. A vertebral treatment system and methods of use are also provided.

### CROSS REFERENCE TO RELATED APPLICATIONS

This patent application claims the benefit of U.S. Provisional Patent Application Ser. No. 60/809,945, filed on Jun. 1, 2006, the contents of which being incorporated herein by reference in its entirety.

### BACKGROUND OF THE INVENTION

A. Field of the Invention

The present disclosure relates to medical devices, components, and methods of use, such as vertebral treatment devices, fluid transfer devices, bone drills, bone drill assemblies, and bone cavity creation/enlargement devices, especially for treating vertebral body and sacral fractures, as well as lytic (destructive) tumor deposits in bone.

B. Background Information

Throughout the years and most recently in particular, various instruments have been developed for use in and for particular medical procedures and/or techniques requiring bone access and treatment. In some bone access procedures, it is necessary to create one or more holes in a bone or bone sections or to bore through the bone. Medical instruments known as bone drills have been developed for creating such holes and bores. Other instruments such as catheters, needles, guide needles, curettes and the like may then be introduced into the hole. On occasion, a cavity needs to be created or enlarged to facilitate treatment of a bone lesion. In some cases, artificial materials may be introduced into the vertebral body, such as cement into the cavity created in the bone, to treat vertebral or sacral fractures.

Medical procedures that require drilling into bone often require creating a cavity or enlarging a cavity in the bone. Examples of such medical procedures include vertebroplasty and/or vertebral augmentation procedures, sacroplasty, and osteoplasty.

Vertebral augmentation and vertebroplasty are procedures for treating vertebral compression fractures. Sacroplasty is a procedure for treating sacral fractures. Osteoplasty is a procedure for treating painful lytic (destructive) tumor deposits in bone. Osteoporosis is a common cause for vertebral compression fractures and sacral fractures, however, tumors involving the spine such as multiple myeloma and metastatic disease can also cause these fractures. A vertebral body compression fracture (VCF) is a fracture involving the vertebral body, which causes the vertebral body to be compressed or collapse. This can lead to shortening and tilting of the spinal column with a forward curvature. This forward curvature can lead to pulmonary and gastrointestinal complications. These fractures are extremely painful and debilitating with many of these patients needing wheelchairs for less painful ambulation; many of these patients are bed-ridden.

Vertebral augmentation and vertebroplasty are designed to stabilize VCFs and relieve pain. Vertebral augmentation also offers the benefit of potential vertebral height restoration and deformity reduction.

In vertebral augmentation and vertebroplasty, access needles are manually pushed or hammered into the fractured vertebral body using fluoroscopic (X-ray) guidance. Various instruments such as a curette may then be inserted through the access needles or tubes. At that point in vertebroplasty, an orthopedic bone filler/cement (e.g. PMMA) is instilled into the fractured bone. In vertebral augmentation, before the bone cement is instilled, balloon catheters are inserted through the access needles or tubes into the fractured vertebral body. The balloon catheters are inflated in an attempt to restore the compressed/collapsed vertebral body to its original height and also to create a cavity in the fractured bone. Following the balloon dilation, the balloons are removed and thicker bone cement instilled into the fractured vertebral body through the access needles or tubes. The cement hardens quickly for both procedures, providing strength and stability to the vertebra. The progress of both procedures is continually monitored in real time with fluoroscopic (X-ray) guidance.

In sacroplasty, access needles are manually pushed or hammered into the fractured sacrum using fluoroscopic (X-ray) or computed tomographic (CT) guidance. Various instruments such as curettes or balloons may then be inserted through the access needles. An orthopedic bone filler/cement (e.g. PMMA) is then instilled through the access needles/tubes into the fractured sacrum. This has been found to provide pain relief and stability. Procedural progress is continually monitored with CT and/or fluoroscopic guidance.

In osteoplasty, access needles are manually pushed or hammered into the lytic (destructive) bone tumor deposit using fluoroscopic (X-ray) or computed tomographic (CT) guidance. Various instruments such as curettes, balloons, or radiofrequency (RF) probes may be inserted through the access needles. An orthopedic bone filler/cement (e.g.) PMMA is then instilled through the access needles/tubes into the lytic deposit. This has been found to provide pain relief and stability. Procedural progress is continually monitored with CT and/or fluoroscopic guidance. It has been recognized, however, that filler, such as the cement for the treatment procedures described above, can flow out through cracks in the targeted bone into undesired areas adjacent to the targeted bone such as the intervertebral disc, spinal canal, neural foramina, or blood vessels. This disadvantageously can result in undesirable health risks to a patient.

Therefore, it would be desirable to overcome the disadvantages and drawbacks of the prior art with improved vertebral treatment devices and related methods of use. It would be desirable if the vertebral treatment device and methods disclosed include an improved fluid injection device that instills artificial materials, such as cement, into targeted bone to treat maladies of the vertebral body and sacrum. It would be highly desirable if such a fluid injection device reduced leakage of filler materials from a bone and the consequent health risks to a patient. It would also be desirable if a vertebral treatment system is provided, which includes a bone drill and/or a cavity drill. Desirably, the vertebral treatment system has a forceps that facilitates guidance and stability during a drilling procedure.

### SUMMARY OF THE INVENTION

Accordingly, an improved vertebral treatment device and related methods of use are provided for overcoming the disadvantages and drawbacks of the prior art. Desirably, the vertebral treatment device and methods disclosed include an improved fluid/transfer injection device that instills artificial materials, such as cement, into targeted bone to treat maladies of the vertebral body and sacrum. The fluid/transfer injection device may be advantageously employed to reduce leakage of filler materials from a bone and minimize the consequent health risks to a patient. It would also be desirable if a vertebral treatment system is provided. Desirably, the vertebral/bone treatment system has a largely radiolucent, off-angle bone drill, cavity creation/enlargement device as well as a forceps that facilitates guidance and stability during a drilling procedure.

In one particular embodiment, in accordance with the principles of the present disclosure, a fluid transfer device is provided, which is adapted to treat a vertebral body, sacrum, or other bony lesion. The fluid transfer device includes a first cavity having a first plunger disposed therewith. The first plunger is configured to draw a first fluid into the first cavity. A second cavity has a second plunger disposed therewith. The second plunger is configured to expel a second fluid from the second cavity. An actuator is connected to the first plunger and the second plunger. The fluid transfer device may have a pressure gauge and automatic stop designed to prevent excessive pressure buildup in the targeted bone and to minimize the risk of cement/filler/fluid leak.

The configuration of the fluid transfer device creates a preferred pathway between the second cavity, such as, for example, a delivery syringe and bony cavities to improve the flow of filler/cement in order to get more uniform filling/distribution of the filler/cement throughout the targeted bone; the configuration also minimizes undesired filler/cement leak into undesired adjacent structures such as the inter-vertebral disc, spinal canal, neural foramina, and blood vessels.

The fluid transfer device may further include a body having a first cylinder. The first cylinder defines the first cavity and supports the first plunger. The body may have a second cylinder, which defines the second cavity and supports the second plunger. The first cavity and the second cavity can communicate with a vertebral cavity of the vertebral body. The actuator may be operatively coupled to the first plunger and the second plunger via a gearing assembly.

The first plunger can include a shaft having teeth axially disposed therealong. The teeth engage the gearing assembly to facilitate movement of the first plunger. The second plunger may include a shaft having teeth axially disposed therealong. The teeth of the second plunger engages the gearing assembly to facilitate movement of the second plunger. The actuator may be rotatable such that the gearing assembly engages the shafts of the plungers to facilitate movement thereof. The body may include a handle.

The gearing assembly can include a first pinion gear that engages the teeth of the first plunger and a second pinion gear that engages the teeth of the second plunger. The pinion gears engage a gear that is operatively connected to the actuator.

In an alternate embodiment, a method for treating a vertebral body having a vertebral cavity is disclosed. The method includes the steps of: providing a fluid transfer device; and simultaneously withdrawing a first fluid from the vertebral cavity and instilling cement into the vertebral cavity.

In another alternate embodiment, a vertebral treatment system is provided. The vertebral treatment system includes a bone drill configured for treating bone of a vertebral body. The bone drill includes a handle connected to a drive housing. The drive housing is connected to a head portion. The head portion includes a shaft extending therefrom. The shaft includes a drill bit and a sheath disposed about the drill bit. The shaft is coupled to a motor disposed with the drive housing via gearing such that the motor rotates the drill bit and the sheath. A fluid transfer device is provided, similar those described herein.

Alternatively, the vertebral treatment system may further include a cavity drill having a body with a sheath extending therefrom and being mounted with the bone drill. The body supporting gearing that operatively couples the sheath to a motor of the bone drill for rotation of the sheath. The vertebral treatment system may further include a forceps. The head portion of the bone drill may include radio opaque markers disposed in a configuration to facilitate alignment of the shaft during a fluoroscopy procedure.

The various aspects of the present disclosure will be more apparent upon reading the following detailed description in conjunction with the accompanying drawings.

### BRIEF DESCRIPTION OF THE DRAWINGS

The above-mentioned and other features and objects of this disclosure, and the manner of attaining them, will become more apparent and the disclosure itself will be better understood by reference to the following description of embodiments of the disclosure taken in conjunction with the accompanying drawings, wherein:

FIG. 1 is a perspective view of one particular embodiment of a vertebral treatment device constructed in accordance with the principles of the present disclosure;

FIG. 2 is a rear perspective view of the vertebral treatment device shown in FIG. 1;

FIG. 3 is a front perspective view of the vertebral treatment device shown in FIG. 1;

FIG. 4 is a front perspective view of the vertebral treatment device shown in FIG. 1, with the cylinder bodies removed;

FIG. 5 is a top perspective view of the vertebral treatment device shown in FIG. 1, with a body portion removed;

FIG. 6 is a rear perspective view of the vertebral treatment device shown in FIG. 1, with the body portion removed;

FIG. 7 is a perspective view of a bone drill constructed in accordance with the principles of the present disclosure;

FIG. 8 is a perspective view of an alternate embodiment of the bone drill shown in FIG. 11 constructed in accordance with the principles of the present disclosure;

FIG. 9 is a perspective view of a device employed in a vertebral treatment procedure constructed in accordance with the principles of the present disclosure; and

FIG. 10 is a diagram of a procedure employing the vertebral treatment device shown in FIG. 1.

Like reference numerals indicate the similar parts throughout the figures.

### DETAILED DESCRIPTION OF EMBODIMENTS OF THE INVENTION

The exemplary embodiments of the vertebral treatment device and methods of use disclosed are discussed in terms of medical apparatus and more particularly, in terms of vertebral treatment devices, fluid transfer devices, bone drills, bone drill assemblies and bone cavity drills that can be employed for treating vertebral body and sacral fractures. The vertebral treatment devices may also be employed to treat lytic tumor deposits in bone. It is envisioned that the present disclosure may be employed with a range of applications including vertebral augmentation, vertebroplasty, sacroplasty and osteoplasty. It is further envisioned that the present disclosure may be used with other medical applications such as diagnosis, treatment and surgery.

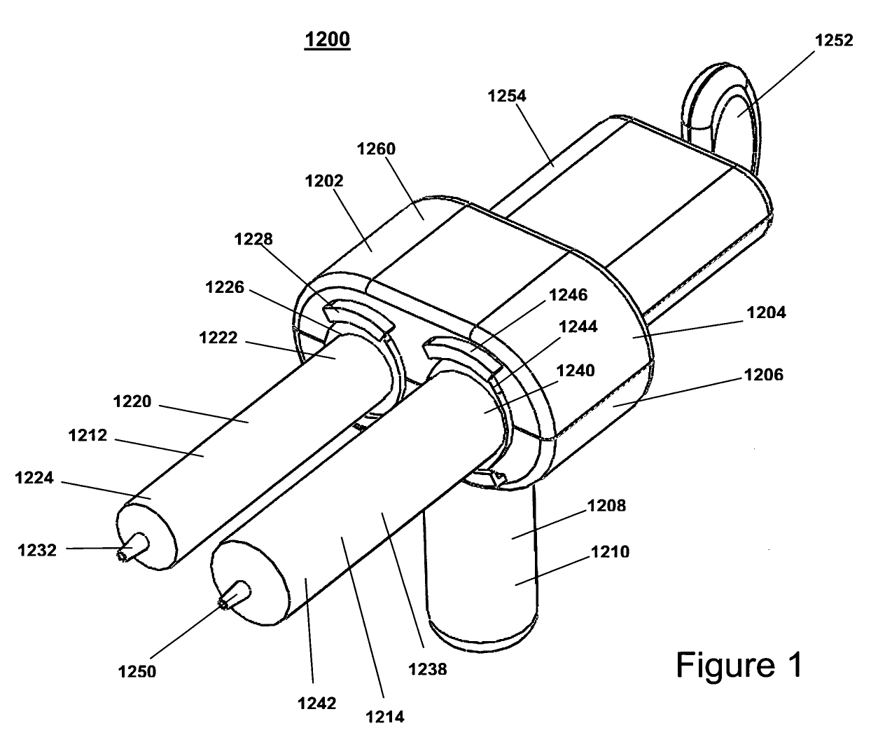

The following discussion includes a description of the vertebral treatment devices, related components and exemplary methods of operating the vertebral treatment devices in accordance with the principles of the present disclosure. Alternate embodiments are also disclosed. Reference will now be made in detail to the exemplary embodiments of the present disclosure, which are illustrated in the accompanying figures. Turning now to FIG. 1, there is illustrated a vertebral treatment device, such as, for example, a fluid transfer device 1200, in accordance with the principles of the present disclosure.

The components of fluid transfer device 1200 are fabricated from materials suitable for medical applications, such as, for example, polymerics and/or metals, depending on the particular application and/or preference. Semi-rigid and rigid polymerics are contemplated for fabrication, as well as resilient materials, such as molded medical grade polyurethane, etc. It is contemplated that any motors, gearing, electronics and power components employed with fluid transfer device 1200 may be fabricated from those suitable for a medical application. Fluid transfer device 1200 may also include circuit boards, circuitry, processor components, etc. for computerized control. One skilled in the art, however, will realize that other materials and fabrication methods suitable for assembly and manufacture, in accordance with the present disclosure, also would be appropriate.

Detailed embodiments of the present disclosure are disclosed herein, however, it is to be understood that the described embodiments are merely exemplary of the disclosure, which may be embodied in various forms. Therefore, specific functional details disclosed herein are not to be interpreted as limiting, but merely as a basis for the claims and as a representative basis for teaching one skilled in the art to variously employ the present disclosure in virtually any appropriately detailed embodiment.

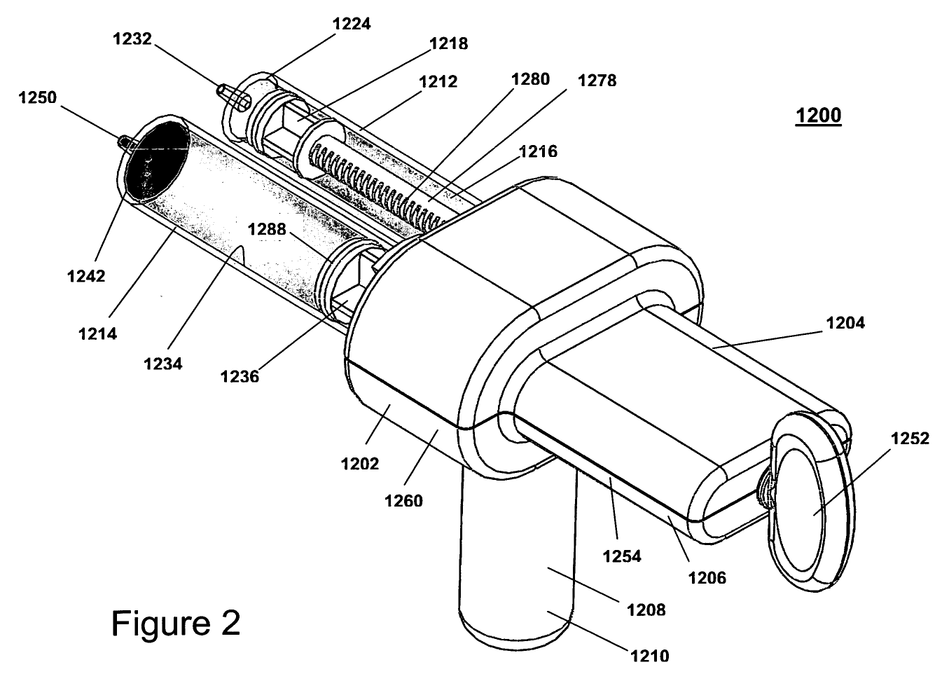

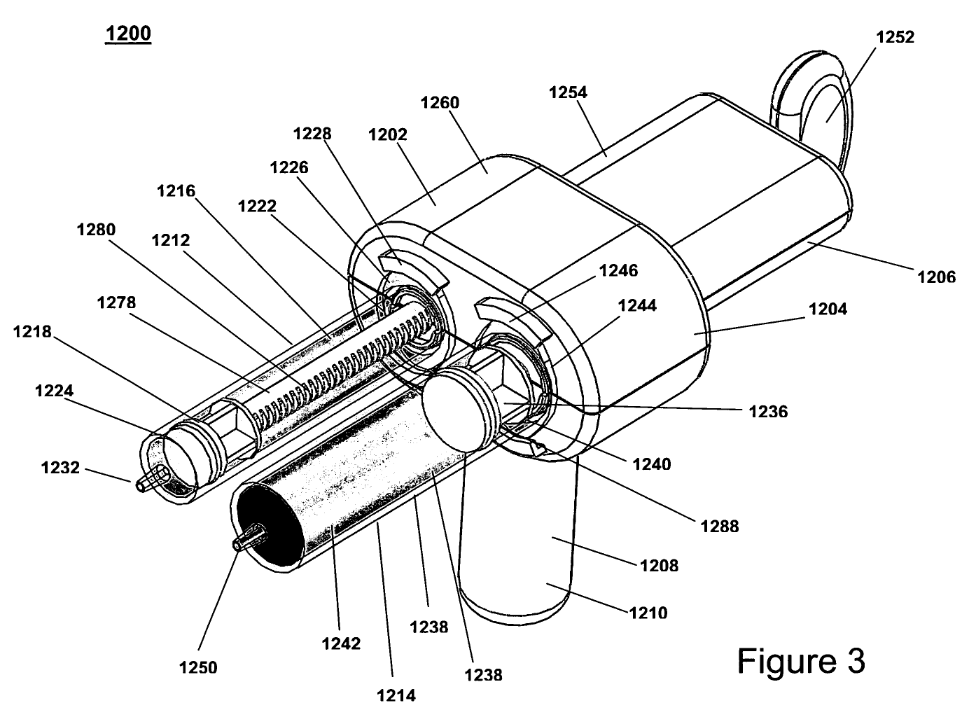

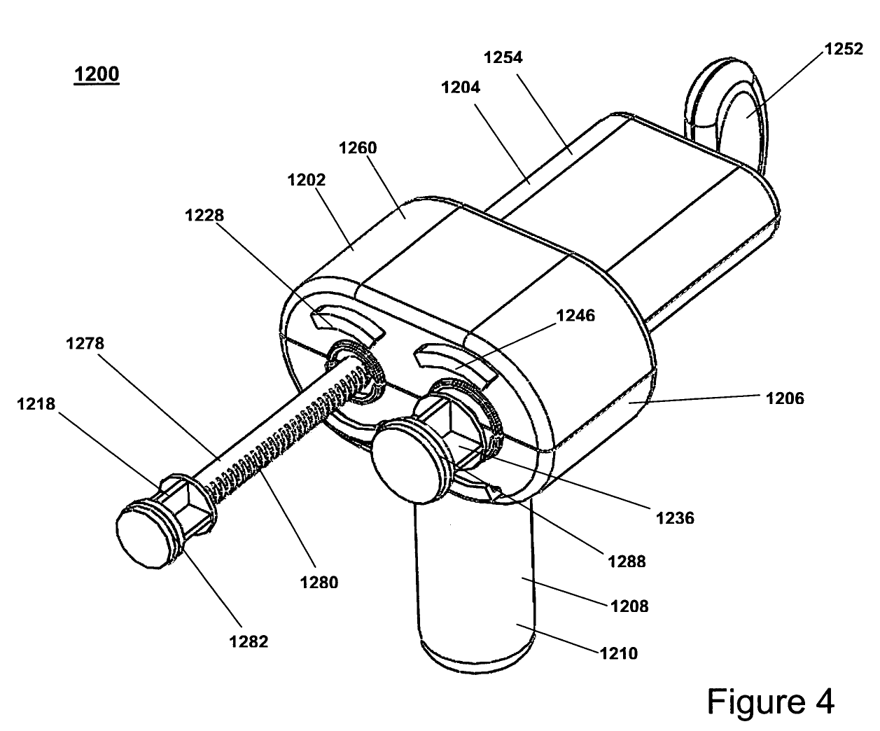

Referring to FIGS. 1-6, fluid transfer device 1200 is adapted for treating vertebral and sacral fractures to facilitate, for example, instilling orthopedic bone filler/cement into fractured bone. Various components, as desired, of fluid transfer device 1200 may be formed of a radio translucent (radiolucent) material and/or radioopaque material. Fluid transfer device 1200 is adapted to treat a vertebral body having a vertebral cavity, as will be discussed. It is envisioned that fluid transfer device 1200, or components thereof are disposable after a vertebral body or sacral body procedure. Fluid transfer device 1200 and its components may also be reused.

Fluid transfer device 1200 has a body 1202. Body 1202 includes a first section 1204, a second section 1206 and a handle 1208. First section 1204 and second section 1206 are integrally assembled to support components of fluid transfer device 1200. It is contemplated that first section 1204 and second section 1206 may be adhered as a unit, retained by mechanical structure such as, clips, pins, etc., or by other methods known to one skilled in the art. Portions of body 1202 may be monolithically formed. It is further contemplated that sections 1204, 1206 may be symmetric halves, offset, non-symmetric, etc.

Handle 1208 is connected with second section 1206 and facilitates grasping/manipulation of fluid transfer device 1200 by a user. Handle 1208 has a tubular body 1210, which is configured for a user's hand to wrap around. It is envisioned that tubular body 1210 may include a finger grip area. Tubular body 1210 may also be pivotable relative to body 1202 to facilitate manipulation and use of fluid transfer device 1200 for a particular application, as well as protecting a user from radiation during fluoroscopy.

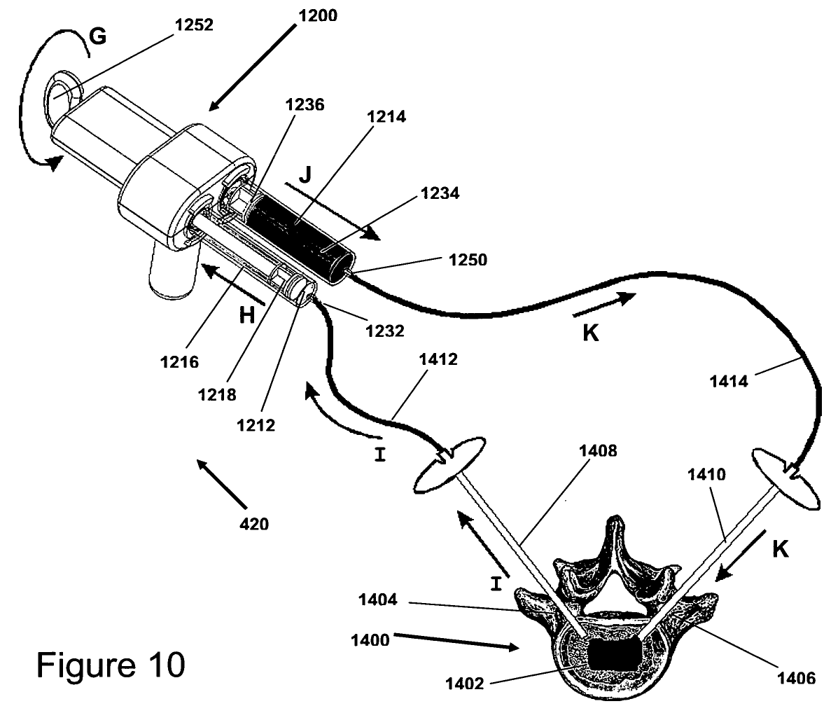

Body 1202 supports a first cylinder, such as, for example, syringe 1212 and a second cylinder, such as, for example, syringe 1214. Syringe 1212 defines a first cavity 1216 and supports a first plunger 1218. First plunger 1218 is disposed for axial movement within first cavity 1216 in a configuration such that a first fluid, such as, for example, body fluid, including blood and/or bone fragments, is drawn into first cavity 1216 from a vertebral cavity of a vertebral body (FIG. 10).

Syringe 1212 has a tubular body 1220 that extends from a first end 1222 to a second end 1224. First end 1222 includes a flange 1226, which is releasably mounted with body 1202 via brackets 1228. Flange 1226 has tabs 1230 that can be rotated into and out of position with brackets 1228 for retaining and releasing syringe 1212 from body 1202. It is contemplated that syringe 1212 may also be releasably mounted with body 1202 by other structure such as a spring mechanism, insertion with body 1202, clips, threaded engagement, luer lock, etc. It is further contemplated that syringe 1212 may be permanently fixed with body 1202 via adhesive, locking assembly, monolithically formed components, etc. It is envisioned that tubular body 1220 may be variously configured and dimensioned accordingly to the requirements of a particular application including alternative cross section such as elliptical, polygonal, etc.

Second end 1224 has a nozzle 1232 configured to receive the body fluid being drawn from the vertebral cavity. Nozzle 1232 is connected to an access tube or the like, discussed below, which is a conduit for the drawn body fluid. It is contemplated that nozzle 1232 and its opening may be variously configured and dimensioned accordingly to the requirements of a particular application including alternative opening sizes to regulate fluid flow.

Syringe 1214 defines a second cavity 1234 and supports a second plunger 1236. Second plunger 1236 is disposed for axial movement within second cavity 1234 in a configuration such that a second fluid, such as, for example, an orthopedic bone filler/cement, or other type of desirable medication/material is, for example, instilled into a fractured bone for treating vertebral and sacral fractures, as will be discussed.

Syringe 1214 has a tubular body 1238 that extends from a first end 1240 to a second end 1242. First end 1240 includes a flange 1244, which is releasably mounted with body 1202 via brackets 1246. Flange 1244 has tabs 1248 that can be rotated into and out of position with brackets 1246 for retaining and releasing syringe 1214 from body 1202. It is contemplated that syringe 1214 may also be releasably mounted with body 1202 by other structure such as a spring mechanism, insertion with body 1202, clips, threaded engagement, luer lock, etc. It is further contemplated that syringe 1214 may be permanently fixed within the body 1202 via adhesive, locking assembly, monolithically formed components, etc. It is envisioned that tubular body 1238 may be variously configured and dimensioned accordingly to the requirements of a particular application including alternative cross section such as elliptical, polygonal, etc.

Second end 1242 has a nozzle 1250 configured to expel the bone filler/cement to the vertebral cavity. Nozzle 1250 is connected to an access tube or the like, discussed below, which is a conduit for the bone filler/cement to the vertebral cavity. It is contemplated that nozzle 1250 and its opening may be variously configured and dimensioned accordingly to the requirements of a particular application including alternative opening sizes to regulate fluid flow.

An actuator 1252 is supported by a rearward portion 1254 of body 1202. Actuator 1252 includes a shaft 1256, which is operatively coupled to a gearing assembly 1258. Gearing assembly 1258 is supported by a forward portion 1260 of body 1202. Actuator 1252 is connected to first plunger 1218 and second plunger 1236 via gearing assembly 1258. It is contemplated that actuator 1252 may be directly connected to plungers 1218, 1236 using, for example, an axial force to move the plungers. It is further contemplated that a plurality of actuator may be employed to facilitate movement of the plungers, such as dedicated actuator for each plunger.

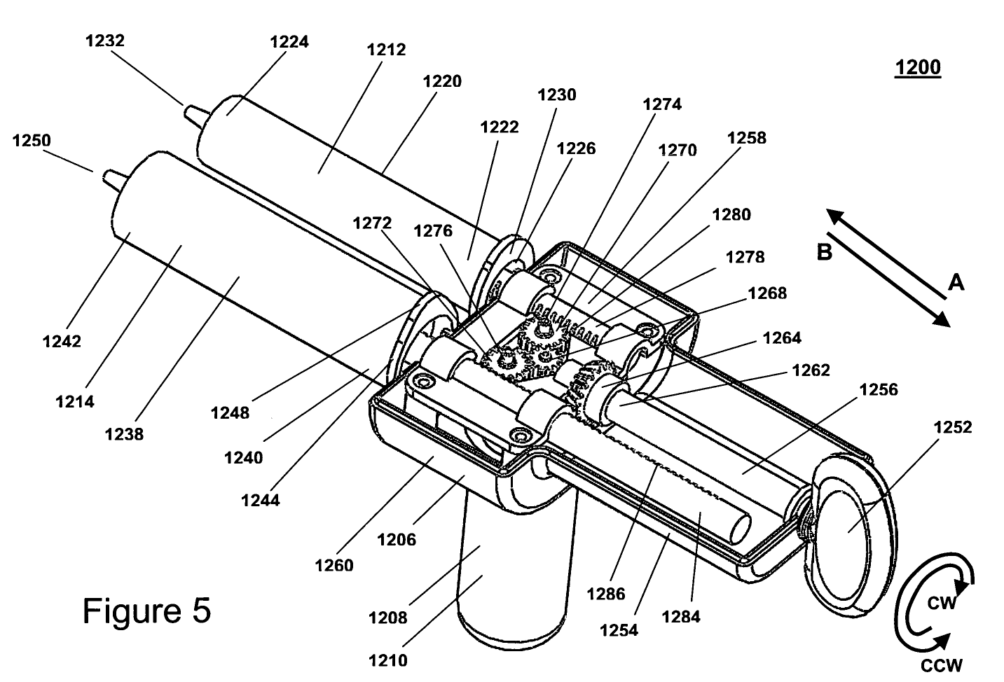

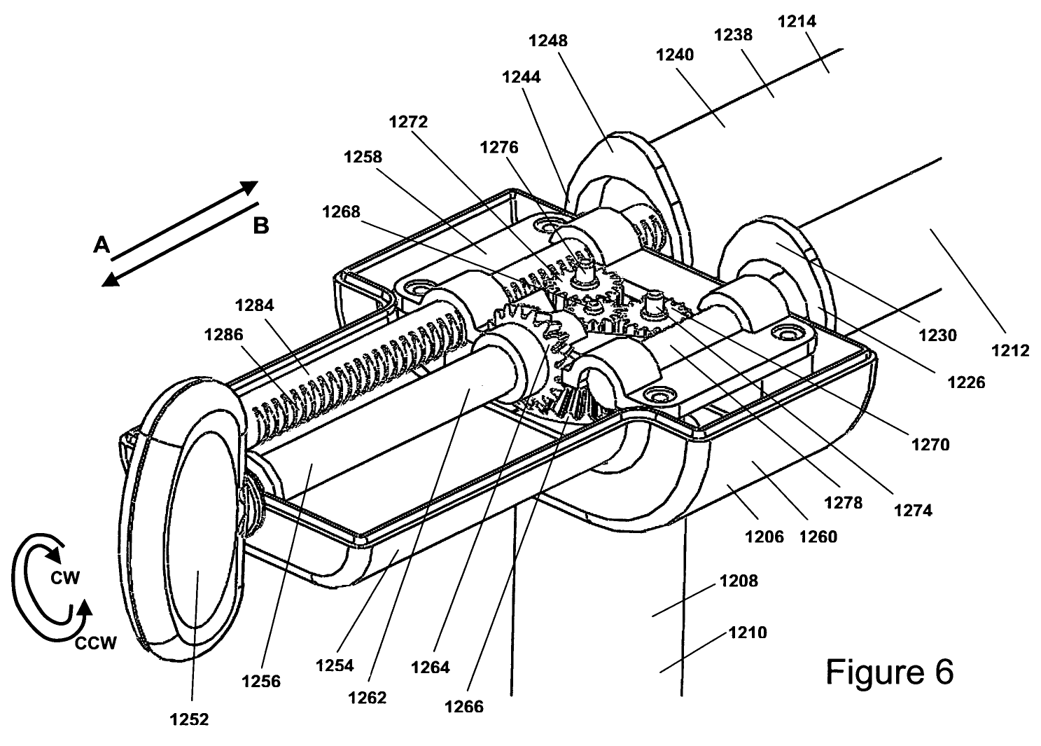

As shown in FIGS. 5 and 6, shaft 1256 extends to an end 1262, which has a bevel gear 1264 mounted thereto. Bevel gear 1264 is operatively coupled to a wheel gear 1266 having teeth disposed on an outer radial periphery thereof. The teeth of wheel gear 1266 mesh/engage with the teeth of bevel gear 1264. As actuator 1252 is manipulated for rotation in a particular direction, for example, clockwise or counterclockwise, shaft 1256 rotates bevel gear 1264 in a corresponding direction. In turn, bevel gear 1264 meshes with wheel gear 1266 to cause rotation thereof. Clockwise rotation of actuator 1252 causes clockwise rotation of wheel gear 1266. Counterclockwise rotation of actuator 1252 causes counterclockwise rotation of wheel gear 1266. Rotation of wheel gear 1266 causes corresponding and simultaneous rotation of a gear 1268, which is mounted with wheel gear 1266. Gear 1268 meshes/engages with a pinion gear 1270 and a pinion gear 1272, which rotate on respective shafts 1274, 1276 mounted with gearing assembly 1258.

Plunger 1218 includes a shaft 1278 having teeth 1280 axially disposed therealong. Teeth 1280 engage pinion gear 1270 to facilitate movement of first plunger 1218 relative to body 1220. For example, as actuator 1252 is manipulated for clockwise rotation, in the direction shown by arrow C, gear 1268 rotates in a clockwise direction. Gear 1268 causes pinion gear 1270 to rotate in a counterclockwise direction. Pinion gear 1270 meshes with teeth 1280 causing shaft 1278 to move first plunger 1218 within first cavity 1216 in a forward axial direction relative to body 1220, in the direction shown by arrow A. Such clockwise rotation of actuator 1252 causes plunger 1218 to expel a fluid, or prepare for drawing fluid, into first cavity 1216. Plunger 1218 includes a gasket 1282 that sealingly engages the inner wall of body 1220. This configuration establishes a vacuum pathway to create suction or expulsion pressure in the communication between first cavity 1216, the vertebral cavity and second cavity 1234, which includes intermediary access lines, tubing or devices.

Conversely, in an example such that actuator 1252 is manipulated for counterclockwise rotation, in the direction shown by arrow CC, gear 1268 rotates in a counterclockwise direction. Gear 1268 causes pinion gear 1270 to rotate in a clockwise direction. Pinion gear 1270 meshes with teeth 1280 causing shaft 1278 to move first plunger 1218 within first cavity 1216 in a rearward direction relative to body 1220, to draw fluid out of the vertebral cavity, facilitating the flow of filler/cement to the vertebral cavity from syringe 1214.

Plunger 1236 of syringe 1214 includes a shaft 1284 having teeth 1286 axially disposed therealong. Teeth 1286 engage pinion gear 1272 to facilitate movement of second plunger 1236 relative to body 1238. For example, as actuator 1252 is manipulated for clockwise rotation, in the direction shown by arrow C, gear 1268 rotates in a clockwise direction. Gear 1268 causes pinion gear 1272 to rotate in a counterclockwise direction. Pinion gear 1272 meshes with teeth 1286 causing shaft 1284 to move second plunger 1236 within second cavity 1234 in a rearward axial direction relative to body 1238, in the direction shown by arrow B. Such clockwise rotation of actuator 1252 causes plunger 1236 to draw a fluid into second cavity 1234. Plunger 1236 includes a gasket 1288 that sealingly engages the inner wall of body 1238. This configuration establishes a preferred vacuum pathway to create suction or expulsion pressure in the communication between first cavity 1216, the vertebral cavity and second cavity 1234, which includes intermediary access lines, tubing or devices.

Conversely, in an example such that actuator 1252 is manipulated for counterclockwise rotation, in the direction shown by arrow CC, gear 1268 rotates in a counterclockwise direction. Gear 1268 causes pinion gear 1272 to rotate in a clockwise direction. Pinion gear 1272 meshes with teeth 1285 causing shaft 1284 to move second plunger 1236 within second cavity 1234 in a forward direction relative to body 1238, in the direction shown by arrow A. Such counterclockwise rotation of actuator 1252 causes plunger 1236 to expel bone filler/cement out of second cavity 1234. Expulsion of the bone filler/cement is facilitated by the sealing engagement of gasket 1288 with the inner wall of body 1238, as discussed. This configuration facilitates a preferred pathway between second cavity 1234 and the vertebral cavity for the flow of filler/cement to the vertebral cavity from syringe 1214. This advantageously reduces the risk of filler/cement flowing out from the vertebral body. The preferred pathway discussed, prevents leakage of filler/cement and undesired filler/cement flow into adjacent structures such as intervertebral disc, spinal canal, neural foramina, and blood vessels.

Fluid transfer device 1200 includes syringes 1212, 1214 such that their respective plungers 1218, 1236 are linked. As plunger 1236 is driven forward, plunger 1218 is driven rearward, as discussed, to create a suction for drawing fluid out of the vertebral cavity of the vertebral body. This advantageous configuration of fluid transfer device 1200 and the methods described, creates a space in the vertebral cavity and a preferred pathway for instilling the bone filler/cement in the vertebral cavity. It is contemplated that alternative to a user manipulated actuator, plungers 1212, 1214 may be moveable via motors, which may include electronic circuitry and microprocessor control. Such control can be employed to monitor and regulate, via adjustment and calibration, the delivery of the bone filler/cement and pressure.

It is envisioned that fluid transfer device 1200 includes a pressure monitoring gauge (not shown), which is connected to the preferred pathway discussed. The pressure monitoring gauge is employed to monitor pressure in the preferred pathway and is connected to an automatic stop mechanism (not shown). The automatic stop mechanism can be activated to discontinue operation of fluid transfer device 1200 to advantageously prevent buildup of excessive pressure in the targeted bone to minimize the likelihood of filler/cement leak. It is further envisioned that such a pressure monitoring gauge may be connected at other locations of the preferred pathway such as with tubing or other devices employed.

In one method of using fluid transfer device 1200, a bore may be created in bone of a vertebral or sacral body, to introduce and temporarily leave a tube, tubular sheath or the like. A tubular sheath may be used, which is configured to allow an instrument, component, tool or the like to pass therethrough and provide access to an area at or adjacent to the vertebral cavity of the vertebral body. Fluid transfer device 1200 may include radiolucent and radio opaque materials. Fluid transfer device 1200 may also include radio opaque markers for aligning components such as tubing, cannulas, needles, sheaths, etc., during a procedure for treating a vertebral body.

In another particular embodiment, in accordance with the principles of the present disclosure, a vertebral treatment system is provided. The vertebral treatment system includes components such as a bone drill, forceps, a cavity drill and a fluid transfer device for treating fractured bone of a vertebral body and/or a sacral body. It is envisioned that the vertebral treatment system may include one or all of the components discussed herein. It is further envisioned that the vertebral treatment system may include other components applicable to a vertebral treatment procedure and in accordance with the present disclosure.

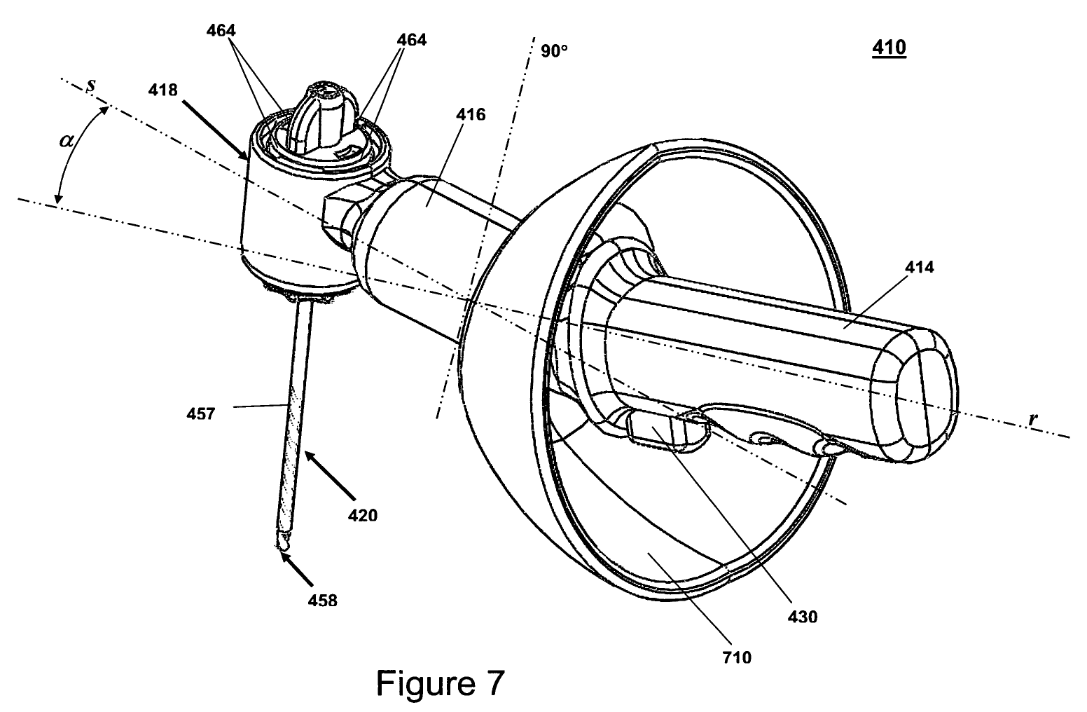

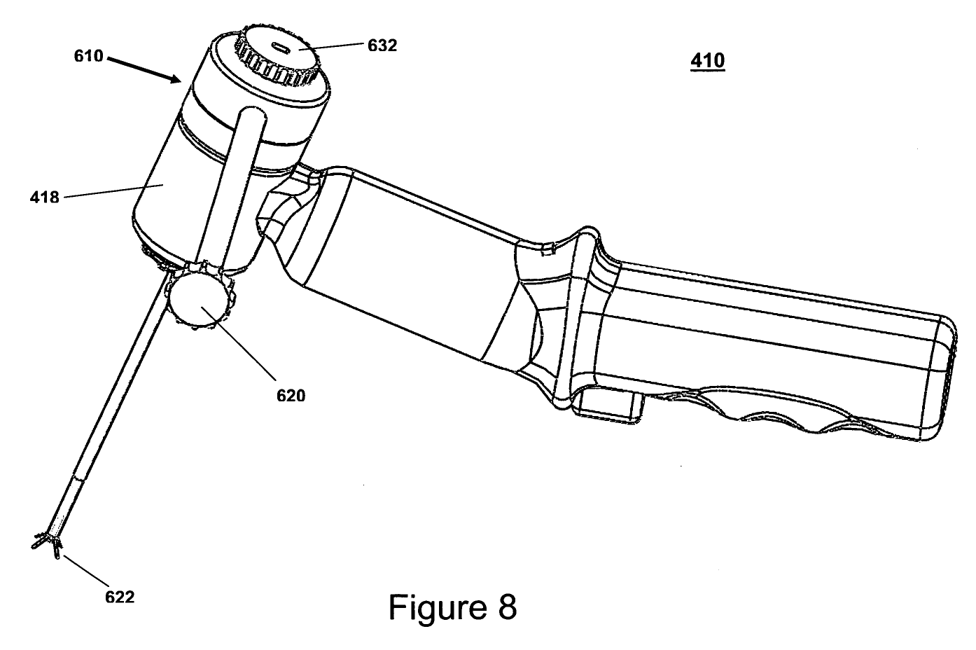

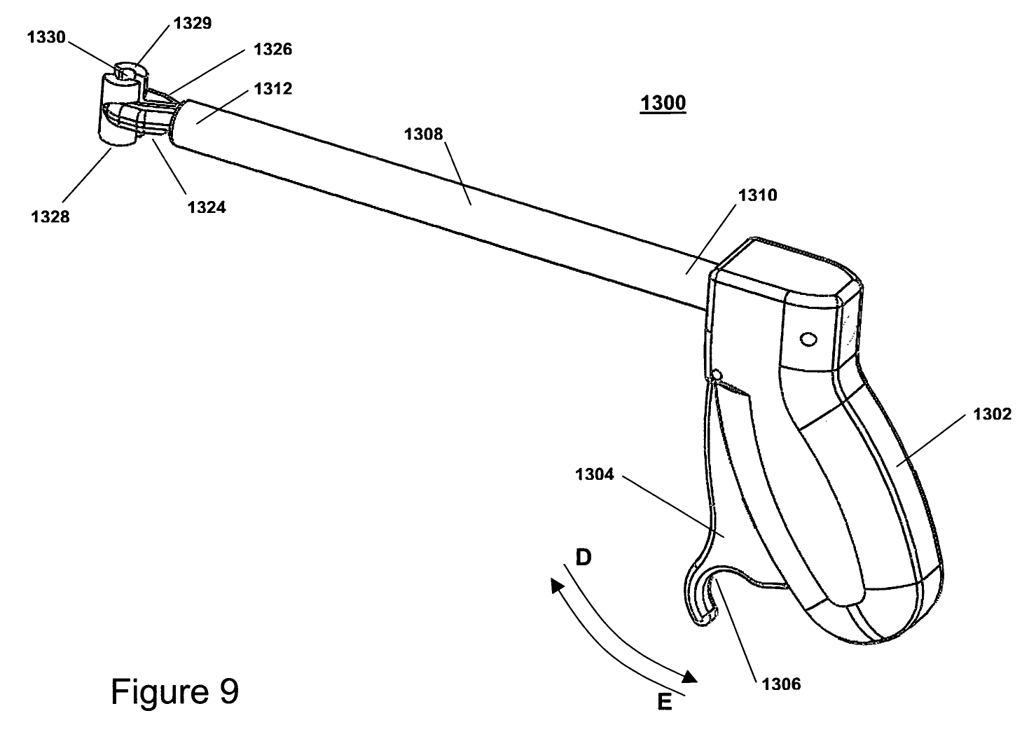

The vertebral treatment system employs, for example, a bone drill 410, as shown in FIG. 7, and a cavity drill 610, as shown in FIG. 8. See, for example, the description of the bone drills and the cavity drills disclosed in co pending and commonly owned U.S. application Ser. No. ______, filed on Apr. 20, 2007 under Express Mail Label No. ER 550793142 US and U.S. application Ser. No. ______, filed on Apr. 20, 2007 under Express Mail Label No. ER 550793139 US, the entire contents of these disclosures being incorporated by reference herein. The vertebral treatment system also employs a fluid transfer device, similar to that described above with regard to FIGS. 1-6, and a forceps 1300, as shown in FIG. 9. See, for example, the description of the forceps disclosed in co pending and commonly owned U.S. application Ser. No. ______, filed on Apr. 20, 2007 under Express Mail Label No. ER 550793156 US, the entire contents of which being incorporated by reference herein. It is envisioned that the vertebral treatment system may employ alternative components. Other uses of the described components of the vertebral treatment system are also contemplated.

In operation of the vertebral treatment system, bone drill 410 is employed with a method for treating fractured bone of a vertebral body or a sacral body. The components of bone drill 410 are fabricated, properly sterilized and otherwise prepared for use. Bone drill 410 is provided with handle portion 414, drive portion 416 and head portion 418 in a configuration that provides a safe distance between a physician and radiation emitted during the procedure.

Head portion 418 includes radioopaque markers 464 disposed in a configuration to facilitate alignment of sheath 457 with bone of the vertebral body (FIG. 13). During fluoroscopy, an area is exposed to radiation, which includes bone drill 410 and the bone of the vertebral body. The exposure of radiation to bone drill 410 and radioopaque markers 464 allows the user to identify the location of sheath 457 and drill bit 458 relative to the targeted bone. This configuration facilitates alignment, via radioopaque markers 464, for cutting the bone while protecting the user by maintaining the offset angular orientation of bone drill 410, discussed above. A guard 710, discussed herein, may also be used during the procedure.

Forceps 1300 is provided to stabilize and guide bone drill 410 during drilling of bone of the vertebral body. Forceps 1300 includes radioopaque arms 1324, 1326 having jaws 1328, 1329. The exposure of radiation to forceps 1300 and radioopaque arms 1324, 1326 allows the user to identify the location of jaws 1328, 1329 relative to sheath 457 and drill bit 458 of bone drill 410. This configuration facilitates guidance for drilling and protects the user from radiation by maintaining the hands of the user a safe distance therefrom.

Arms 1324, 1326 are moveable between a closed position and an open position, as discussed above. When jaws 1328, 1329 are in the open position, sheath 457 is free to rotate. To grasp sheath 457 for guidance and stabilization of bone drill 410 during the vertebral drilling procedure, the user grasps handle 1302 and squeezes on actuator 1304. Shaft 1308 moves to the extended position and jaws 1328, 1329 move to the closed position to grasp sheath 457. Cylindrical cavity 1330 is configured to snugly fit and snap onto sheath 457. Sheath 457 is firmly held in position by forceps 1300, which advantageously operates as a drill guide.

Drill bit 458 engages the bone and rotates via motor 498 to bore a cavity in the bone. Sheath 457 is driven into engagement with the bone to further define the cavity in the bone. After drill bit 458 has reached a desired depth within the targeted bone, according to the requirements of a particular procedure, actuator 1304 of forceps 1300 can release jaws 1328, 1329 from sheath 457. Sheath 457 is free to rotate. If desired, forceps 1300 may be removed from sheath 457.

Cavity drill 610, which is an alternate embodiment of bone drill 410, is provided for enlarging and/or further defining the cavity bored in the bone by bone drill 410. Cavity drill 610 includes a knob 620, which is manipulated for rotation to drive a bone curette 622, which reams the targeted bone and cavity. Cavity drill 610 also includes a knob 632, which is manipulated for rotation to cause relative axial translation of bone curette 622. Knobs 620, 632 are rotated, in cooperation to ream the targeted bone area and further define the targeted bone cavity. It is contemplated that cavity drill 610 may include radioopaque markers to facilitate alignment thereof with the targeted bone.

After the cavity is created in the targeted vertebral bone, according to the requirements for the particular fracture and treatment procedure, the targeted vertebral body or sacral body is treated. See, for example, the description of methods of use in co pending and commonly owned U.S. application Ser. No. ______, filed on Apr. 20, 2007 under Express Mail Label No. ER 550793142 US, U.S. application Ser. No. ______, filed on Apr. 20, 2007 under Express Mail Label No. ER 550793139 US and U.S. application Ser. No. ______, filed on Apr. 20, 2007 under Express Mail Label No. ER 550793156 US. It is contemplated that one or a plurality of cavities may be created to allow for access tubing, cannulas, etc. in the targeted area. It is further contemplated that balloon catheters, etc., may be inserted through the access tubing, cannulas, etc. into the targeted fractured vertebral body. Bone fillers/cement may then be instilled into the bone. It is envisioned that the access tubing, cannulas, etc. may be fabricated from radiolucent material and/or radioopaque material.

A fluid transfer device 1200 is provided for treating a fracture of a vertebral body 1400 having a vertebral cavity 1402, as shown in FIG. 10. Bone drill 410, forceps 1300 and cavity drill 610, as discussed, create drilled access cavities 1404, 1406. Access cannulas 1408, 1410 are inserted for positioning with access cavities 1404, 1406.

Tubing 1412, 1414 are connected with access cannulas 1408, 1410. Tubing 1412 is connected to nozzle 1232 and tubing 1414 is connected to nozzle 1250. Syringes 1212, 1214, tubing 1412, 1414, cannulas 1404, 1406 and vertebral cavity 1402 are in fluid communication to establish a preferred vacuum pathway to create suction and expulsion pressure between syringes 1212, 1214 and vertebral cavity 1402 for treating a fracture of vertebral body 1400.

To instill an orthopedic bone filler/cement such as PMMA (Polymethyl methacrylate) into vertebral cavity 1402, fluid transfer device 1200 draws body fluid out of vertebral cavity 1402 and instills PMMA therein. Actuator 1252 is manipulated for counterclockwise rotation, in the direction shown by arrow G. First plunger 1218 is caused to move within first cavity 1216 in a rearward direction, in the direction shown by arrow H, discussed above. Accordingly, body fluid is drawn out of vertebral cavity 1402, in the direction shown by arrows I.

As actuator 1252 is rotated counterclockwise, second plunger 1236 is caused to move within second cavity 1234 in a forward direction, in the direction shown by arrow J. Accordingly, plunger 1236 expels PMMA out of second cavity 1234, in the direction shown by arrows K, and into vertebral cavity 1402. This advantageous configuration removes body fluid and instills bone filler/cement simultaneously, as facilitated by the preferred communication pathway between syringe 1212 and syringe 1214. This design of the vertebral treatment system and fluid transfer device 1200 has several benefits including increased patient safety by reducing the risk of leakage of bone filler/cement and undesired flow of filler/cement into adjacent structures such as the intervertebral disc, spinal canal, neural foramina, and blood vessels.

The PMMA instilled in vertebral cavity 1402 hardens to provide strength and stability to the vertebra. It is envisioned that the vertebral treatment system employing fluid transfer device 1200 may be continuously monitored using fluoroscopy guidance. It is further envisioned that the vertebral treatment system may be employed with various treatment procedures such as vertebral augmentation, vertebroplasty, sacroplasty, osteoplasty, etc.

While the invention has been illustrated and described in detail in the drawings and foregoing description, the same is to be considered as illustrative and not restrictive in character, it being understood that embodiments have been shown and described and that all changes and modifications that come within the spirit of this invention are desired to be protected.

What is claimed is:

1. A fluid transfer device adapted to treat a vertebral body, the fluid transfer device comprising:
a first cavity having a first plunger disposed therewith, the first plunger being configured to draw a first fluid into the first cavity;
a second cavity having a second plunger disposed therewith, the second plunger being configured to expel a second fluid from the second cavity; and
an actuator being connected to the first plunger and the second plunger.

2. A fluid transfer device as recited in claim 1, further comprising a body having a first cylinder, the first cylinder defining the first cavity and supporting the first plunger.

3. A fluid transfer device as recited in claim 2, wherein the body has a second cylinder, which defines the second cavity and supports the second plunger.

4. A fluid transfer device as recited in claim 1, wherein the first cavity and the second cavity communicate with a vertebral cavity of the vertebral body.

5. A fluid transfer device as recited in claim 1, wherein the actuator is operatively coupled to the first plunger and the second plunger via a gearing assembly.

6. A fluid transfer device as recited in claim 5, wherein the first plunger includes a shaft having teeth axially disposed therealong, the teeth engaging the gearing assembly to facilitate movement of the first plunger.

7. A fluid transfer device as recited in claim 6, wherein the second plunger includes a shaft having teeth axially disposed therealong, the teeth of the second plunger engaging the gearing assembly to facilitate movement of the second plunger.

8. A fluid transfer device as recited in claim 7, wherein the actuator is rotatable such that the gearing assembly engages the shafts of the plungers to facilitate movement thereof.

9. A fluid transfer device adapted to treat a vertebral body having a vertebral cavity, the fluid transfer device comprising:
a body supporting a first cylinder and a second cylinder, the first cylinder defining a first cavity and supporting a first plunger disposed for axial movement within the first cavity such that a first fluid is drawn into the first cavity, the first cavity having a shaft including a plurality of axially disposed teeth,
the second cylinder defining a second cavity and supporting a second plunger disposed for axial movement within the second cavity such that a second fluid is expelled from the second cavity, the second plunger having a shaft including a plurality of axially disposed teeth; and
an actuator being supported by the body and operatively coupled to a gearing assembly supported by the body, the gearing assembly being operatively coupled to the teeth of the first plunger and the second plunger,
wherein the actuator causes movement of the first plunger and the second plunger via the gearing assembly, and the first and second cavities being in communication with the vertebral cavity.

10. A fluid transfer device as recited in claim 9, wherein the body includes a handle.

11. A fluid transfer device as recited in claim 9, wherein the actuator is rotatable.

12. A fluid transfer device as recited in claim 9, wherein the gearing assembly includes a first pinion gear that engages the teeth of the first plunger and a second pinion gear that engages the teeth of the second plunger, the pinion gears engaging a gear that is operatively connected to the actuator.

13. A fluid transfer device as recited in claim 9, wherein the second plunger is configured to instill a bone filler into targeted bone.

14. A method for treating a vertebral body having a vertebral cavity, the method comprising the steps of:
providing a fluid transfer device; and
simultaneously withdrawing a first fluid from the vertebral cavity and instilling cement into the vertebral cavity in a configuration to create a preferred pathway from the fluid transfer device to the vertebral cavity allowing uniform distribution of the cement throughout targeted bone.

15. A vertebral treatment system comprising:
an off-angle, radiolucent bone drill configured for treating bone of a vertebral body, the bone drill including a handle connected to a drive housing, the drive housing being connected to a head portion, the head portion including a shaft extending therefrom, the shaft including a drill bit and a sheath disposed about the drill bit, the shaft being coupled to a motor disposed with the drive housing via gearing such that the motor rotates the drill bit and the sheath; and
a fluid transfer device including a first cavity having a first plunger disposed therewith, the first plunger being configured to draw a first fluid into the first cavity, a second cavity having a second plunger disposed therewith, the second plunger being configured to expel a second fluid from the second cavity, and an actuator being connected to the first plunger and the second plunger.

16. A vertebral treatment system as recited in claim 15, further comprising a cavity drill including a body having a sheath extending therefrom and being mounted with the bone drill, the body supporting gearing that operatively couples the sheath to a motor of the bone drill for rotation of the sheath and cutting blades having radio opaque markers.

17. A vertebral treatment system as recited in claim 15, further comprising a radiolucent forceps including a handle including an actuator pivotably connected therewith, a shaft extending from the handle, and an elongated member extending through the shaft and having a proximal end and a distal end, the proximal end being operatively engageable with the actuator and the distal end including opposing arms configured to grasp.

18. A vertebral treatment system as recited in claim 15, wherein the head portion includes radio opaque markers disposed in a configuration to facilitate alignment of the shaft during a fluoroscopy procedure.

19. A fluid transfer device as recited in claim 1, wherein the second plunger is configured to instill a bone filler into targeted bone.

20. A fluid transfer device as recited in claim 1, wherein the second plunger is configured to instill medication into targeted bone.

21. A fluid transfer device as recited in claim 9, wherein the second plunger is configured to instill medication into targeted bone.

22. A fluid transfer device as recited in claim 1, wherein the first cavity and the second cavity are connected to a vertebral cavity of the vertebral body via a preferred pathway, the fluid transfer device further comprising a pressure monitoring device connected to the preferred pathway.

23. A fluid transfer device as recited in claim 22, wherein the pressure monitoring gauge is connected to a stop mechanism configured for activation to discontinue operation of the fluid transfer device.

24. A fluid transfer device as recited in claim 9, wherein the first cavity and the second cavity are connected to the vertebral cavity via a preferred pathway, the fluid transfer device further comprising a pressure monitoring device connected to the preferred pathway.

25. A fluid transfer device as recited in claim 24, wherein the pressure monitoring gauge is connected to a stop mechanism configured for activation to discontinue operation of the fluid transfer device.
